(19) United States
(12) Patent Application Publication
(10) Pub. No.: US 2025/0360264 A1
(43) Pub. Date: Nov. 27, 2025

(54) DEVICES, SYSTEMS, AND METHODS FOR AUTOMATED OPERATION OF INFUSION PUMPS WITH MOBILE DEVICES

(71) Applicant: Tandem Diabetes Care, Inc., San Diego, CA (US)

(72) Inventor: Geoff Kruse, San Diego, CA (US)

(21) Appl. No.: 19/219,227

(22) Filed: May 27, 2025

Related U.S. Application Data
(60) Provisional application No. 63/651,812, filed on May 24, 2024.

(51) Int. Cl. A61M 5/142; G16H 20/17; G16H 40/63

(52) U.S. Cl. CPC A61M 5/142; G16H 20/17; G16H 40/63; A61M 2005/14208; A61M 2205/3303; A61M 2205/3584; A61M 2205/3592; A61M 2205/502; A61M 2205/582; A61M 2205/583; A61M 2230/005; A61M 2230/201; A61M 2230/63

(57) ABSTRACT

A system for medicament infusion may include an infusion pump and a mobile device associated with a user. The mobile device is configured to determine an online dosing model and an offline model. The infusion pump is configured to determine an operation mode of the infusion pump based on a connection status between the mobile device and the infusion pump. The operation mode comprises an online mode when the infusion pump is connected to the mobile device and an offline mode when the infusion pump is not connected to the mobile device. The infusion pump is also configured to deliver a dose of medicament to the user based on the online dosing model or the offline dosing model, in accordance with a determination that the infusion pump is in an online mode or an offline mode.

### CROSS-REFERENCE TO RELATED APPLICATIONS

This application claims the benefit of U.S. Provisional Application No. 63/651,812, filed May 24, 2024, the entirety of which is incorporated herein by reference.

### TECHNICAL FIELD

The present disclosure relates, generally, to infusion pumps and, more specifically, to automated operation of infusion pumps with mobile devices.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art. One category of such fluid delivery devices includes insulin injecting pumps developed for administering insulin to patients afflicted with type 1, or in some cases, type 2 diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent App. Pub. Nos. 2013/0324928 and 2013/0053816, U.S. Pat. Nos. 8,287,495, 8,573,027, 8,986,253, and 9,381,297, as well as PCT Patent App. No. PCT/US23/82084, each of which is incorporated herein by reference in its entirety.

Ambulatory infusion pumps for delivering insulin or other medicaments can be used in conjunction with blood glucose monitoring systems, such as blood glucose meters (BGMs) and continuous glucose monitors (CGMs). A CGM provides a substantially continuous estimated blood glucose level through a transcutaneous sensor that estimates blood analyte levels, such as blood glucose levels, via the patient's interstitial fluid CGM systems typically consist of a transcutaneously placed sensor, a transmitter, and a monitor.

Ambulatory infusion pumps typically allow the patient or caregiver to adjust the amount of insulin or other medicament delivered, by a basal rate or a bolus, based on blood glucose data obtained by a BGM or a CGM, and in some cases include the capability to automatically adjust such medicament delivery. Some ambulatory infusion pumps may include the capability to interface with a BGM or CGM such as, e.g., by receiving measured or estimated blood glucose levels and automatically adjusting or prompting the user to adjust the level of medicament being administered or planned for administration or, in cases of abnormally low blood glucose readings, reducing or automatically temporarily ceasing or prompting the user temporarily to cease or reduce insulin administration. These portable pumps may incorporate a BGM or CGM within the hardware of the pump or may communicate with a dedicated BGM or CGM via wired or wireless data communication protocols, directly and/or via a device such as a smartphone. One example of integration of infusion pumps with CGM devices is described in U.S. Patent App. Pub. No. 2014/0276419, which is hereby incorporated by reference herein.

As noted above, insulin or other medicament dosing by basal rate and/or bolus techniques could automatically be provided by a pump based on readings received into the pump from a CGM device that is, e.g., external to the portable insulin pump or integrated with the pump as a pump-CGM system in a closed-loop or semi-closed-loop fashion. With respect to insulin delivery, some systems including this feature can be referred to as artificial pancreas systems or dynamic artificial pancreas (DAP) system, because the systems serve to mimic biological functions of the pancreas for patients with diabetes. Such systems are also referred to as automated insulin delivery (AID) systems.

An AID system uses measurements of metabolic signals such as interstitial glucose and user inputs such as carbohydrate entries to determine the optimal amount of insulin to deliver to maintain user blood glucose as close as possible to the euglycemic range. Current AID systems employ algorithms that calculate insulin doses on ambulatory infusion pumps which have limited computation capabilities. In addition, current systems have relied heavily on user inputs such as carb entries into an infusion pump to determine a target dose especially when the infusion pump is not connected to external devices.

### SUMMARY

Embodiments of the present disclosure provide apparatuses and methods for automated insulin delivery with a mobile device and an infusion pump.

In some embodiments, a system for medicament infusion includes an infusion pump and a mobile device associated with a user. The mobile device is configured to obtain an online dosing model for dosing determination, obtain dosing information associated with the user, and generate a dosing instruction for the user based on the online dosing model and the dosing information. The mobile device is also configured to determine a plurality of predicted pump states in a predetermined time period based on the dosing information associated with the user, and generate an offline dosing model for the user in the predetermined time period based on the online dosing model and the plurality of predicted pump states. The offline dosing model may have a smaller size than the online dosing model. The infusion pump is configured to obtain the offline dosing model from the mobile device, store the offline dosing model in a local storage of the infusion pump, and determine, in the predetermined time period, whether the infusion pump is in an online mode when the infusion pump is connected to the mobile device or in an offline mode when the infusion pump is not connected to the mobile device. The infusion pump is also configured to obtain the dosing instruction from the mobile device, and deliver a dose of medicament to the user based on the dosing instruction, in accordance with a determination that the infusion pump is in the online mode in the predetermined time period. In accordance with a determination that the infusion pump is in the offline mode in the predetermined time period, the infusion pump is configured to: determine a current pump state corresponding to one of the plurality of predicted pump states, and deliver a dose of medicament to the user based on the offline dosing model and the current pump state.

In some embodiments, an infusion pump includes a processor and a non-transitory, computer-readable medium storing instructions which, when executed by the processor, cause the infusion pump to perform operations. The operations include: transmitting dosing information associated with a user to a mobile device of the user, obtaining a dosing instruction from the mobile device, and obtaining an offline dosing model from the mobile device. The dosing instruction may be generated for the user based on an online dosing model and the dosing information. The offline dosing model may be generated for the user in a predetermined time period based on the online dosing model and a plurality of predicted pump states. The plurality of predicted pump states may be determined for the predetermined time period based on the dosing information associated with the user. The offline dosing model may have a smaller size than the online dosing model. The operations also include storing the offline dosing model in a local storage of the infusion pump, and determining, in the predetermined time period, whether the infusion pump is in an online mode when the infusion pump is connected to the mobile device or in an offline mode when the infusion pump is not connected to the mobile device. In accordance with a determination that the infusion pump is in the online mode in the predetermined time period, the operations include delivering a dose of medicament to the user based on the dosing instruction. In accordance with a determination that the infusion pump is in the offline mode in the predetermined time period, the operations include: determining a current pump state corresponding to one of the plurality of predicted pump states, and delivering a dose of medicament to the user based on the offline dosing model and the current pump state.

In some embodiments, a computer-implemented method for operation of an infusion pump includes: transmitting dosing information associated with a user to a mobile device of the user, obtaining a dosing instruction from the mobile device, and obtaining an offline dosing model from the mobile device. The dosing instruction may be generated for the user based on an online dosing model and the dosing information. The offline dosing model may be generated for the user in a predetermined time period based on the online dosing model and a plurality of predicted pump states. The plurality of predicted pump states may be determined for the predetermined time period based on the dosing information associated with the user. The offline dosing model may have a smaller size than the online dosing model. The operations also include storing the offline dosing model in a local storage of the infusion pump, and determining, in the predetermined time period, whether the infusion pump is in an online mode when the infusion pump is connected to the mobile device or in an offline mode when the infusion pump is not connected to the mobile device. In accordance with a determination that the infusion pump is in the online mode in the predetermined time period, the operations include delivering a dose of medicament to the user based on the dosing instruction. In accordance with a determination that the infusion pump is in the offline mode in the predetermined time period, the operations include: determining a current pump state corresponding to one of the plurality of predicted pump states, and delivering a dose of medicament to the user based on the offline dosing model and the current pump state.

In some embodiments, a non-transitory, computer-readable medium stores instructions which, when executed by a processor of an electronic device, cause the electronic device to perform operations. The operations include: transmitting dosing information associated with a user to a mobile device of the user, obtaining a dosing instruction from the mobile device, and obtaining an offline dosing model from the mobile device. The dosing instruction may be generated for the user based on an online dosing model and the dosing information. The offline dosing model may be generated for the user in a predetermined time period based on the online dosing model and a plurality of predicted pump states. The plurality of predicted pump states may be determined for the predetermined time period based on the dosing information associated with the user. The offline dosing model may have a smaller size than the online dosing model. The operations also include storing the offline dosing model in a local storage of the electronic device, and determining, in the predetermined time period, whether the electronic device is in an online mode when the electronic device is connected to the mobile device or in an offline mode when the electronic device is not connected to the mobile device. In accordance with a determination that the electronic device is in the online mode in the predetermined time period, the operations include delivering a dose of medicament to the user based on the dosing instruction. In accordance with a determination that the electronic device is in the offline mode in the predetermined time period, the operations include: determining a current pump state corresponding to one of the plurality of predicted pump states, and delivering a dose of medicament to the user based on the offline dosing model and the current pump state.

In some embodiments, a mobile device comprises a processor and a non-transitory, computer-readable medium storing instructions which, when executed by the processor, cause the mobile device to perform operations. The operations include: obtaining an online dosing model for dosing determination, and obtaining dosing information from an infusion pump that is configured for delivering medicament to a user associated with the mobile device. The dosing information indicates, in a predetermined time period, whether the infusion pump is in an online mode when the infusion pump is connected to the mobile device or in an offline mode when the infusion pump is not connected to the mobile device. The operations also include: generating a dosing instruction for the user based on the online dosing model and the dosing information, determining a plurality of predicted pump states in the predetermined time period based on the dosing information associated with the user, and generating an offline dosing model for the user in the predetermined time period based on the online dosing model and the plurality of predicted pump states, wherein the offline dosing model has a smaller size than the online dosing model. Further, the operations include transmitting the dosing instruction and the offline dosing model to the infusion pump. The infusion pump is configured to store the offline dosing model in a local storage of the infusion pump. In accordance with a determination that the infusion pump is in the online mode in the predetermined time period, the infusion pump is configured to deliver a dose of medicament to the user based on the dosing instruction. In accordance with a determination that the infusion pump is in the offline mode in the predetermined time period, the infusion pump is configured to: determine a current pump state corresponding to one of the plurality of predicted pump states, and deliver a dose of medicament to the user based on the offline dosing model and the current pump state.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention may be more completely understood in consideration of the following detailed description of various embodiments of the invention in connection with the accompanying drawings, in which:

FIG. 1 is a medical device that can be used with embodiments of the disclosure, according to various embodiments of the present disclosure.

FIG. 2 is a block diagram representing a medical device that can be used with embodiments of the disclosure, according to various embodiments of the present disclosure.

FIGS. 3A-3B depict an embodiment of a pump system, according to various embodiments of the present disclosure.

FIG. 4 is a schematic representation of a glucose monitoring system, according to various embodiments of the present disclosure.

FIG. 5 is a network environment configured for automated insulin delivery, according to various embodiments of the present disclosure.

FIG. 6A is a block diagram of a system for employing automated insulin delivery with a user device and an infusion pump, according to various embodiments of the present disclosure.

FIG. 6B illustrates exemplary look-up tables used for automatically determining a dose of medicament, according to various embodiments of the present disclosure.

FIG. 7 is a flow chart of a method for delivering optimal insulin doses, according to various embodiments of the present disclosure.

FIG. 8A is a graph of a policy, according to various embodiments of the present disclosure.

FIG. 8B is a graph of the policy of FIG. 8A with a flat portion, a quick rise portion, and a slow rise portion identified, according to various embodiments of the present disclosure.

FIG. 8C is a graph of the policy of FIG. 8A with a max flat glucose and max dose identified, according to various embodiments of the present disclosure.

FIG. 8D is a graph of the policy of FIG. 8A with indications of the impacts of adjusting automated glycemic control algorithm parameters identified, according to various embodiments of the present disclosure.

FIG. 9 illustrates optimal insulin dosing targets, according to various embodiments of the present disclosure.

FIG. 10 is a graph showing different policies corresponding to different meal states, according to various embodiments of the present disclosure.

FIG. 11 is a flow chart of a method performed by a mobile device for automatically delivering a dose of medicament, according to various embodiments of the present disclosure.

FIG. 12 is a flow chart of a method performed by an infusion pump for automatically delivering a dose of medicament, according to various embodiments of the present disclosure.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

One objective of the present teaching is to improve the ability of an automated insulin delivery system for delivering insulin to a user using an infusion pump, by shifting at least some computational load from the infusion pump to a mobile device of the user. In some embodiments, the infusion pump may operate in an online mode when the infusion pump is connected to the mobile device, or operate in an offline mode when the infusion pump is not connected to the mobile device. When the infusion pump is connected to the mobile device, the mobile device can compute both an online dosing model to be used in the online mode and an offline dosing model to be used in the offline mode. The offline dosing model may be generated for the user based on the online dosing model and a plurality of predicted pump states in a predetermined time period. The offline dosing model may have a smaller size than the online dosing model and can be stored in a local storage of the infusion pump. As such, when the infusion pump is not connected to the mobile device, the infusion pump falls back to the offline mode to make use of some precomputed dosing functions to deliver a dose of insulin to the user based on a current pump state that is closest to one of the predicted pump states, without a need to re-compute insulin dose from scratch. In various embodiments, the disclosed method can be applied to any infusion pump for delivering a dose of any medicament.

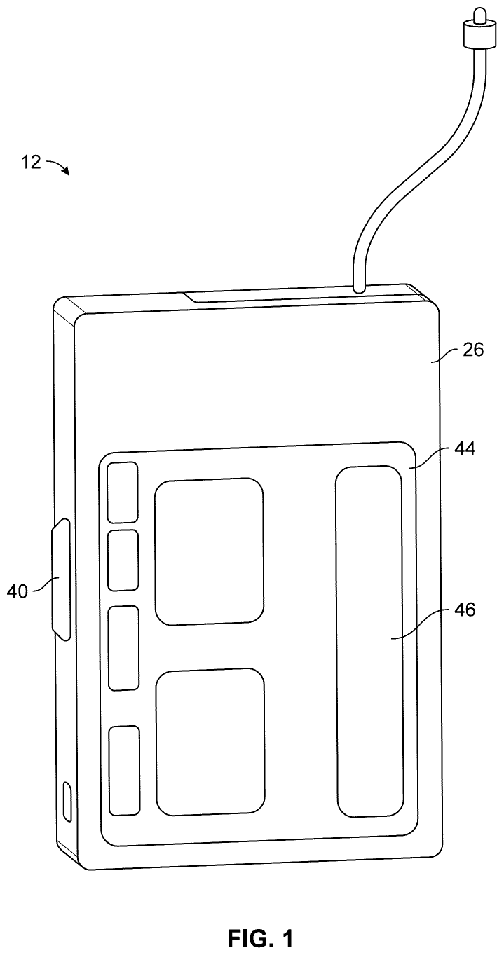

FIG. 1 depicts an embodiment of a medical device according to the disclosure. In this embodiment, the medical device is configured as a pump 12. Pump 12 may be an infusion pump that includes a pumping or delivery mechanism and reservoir for delivering medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display, a remote-control device, a laptop computer, personal computer, tablet computer, a mobile communication device such as a smartphone, a wearable electronic watch or electronic health or fitness monitor, or personal digital assistant (PDA), a CGM display etc.

In one embodiment, the medical device can be an ambulatory insulin pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

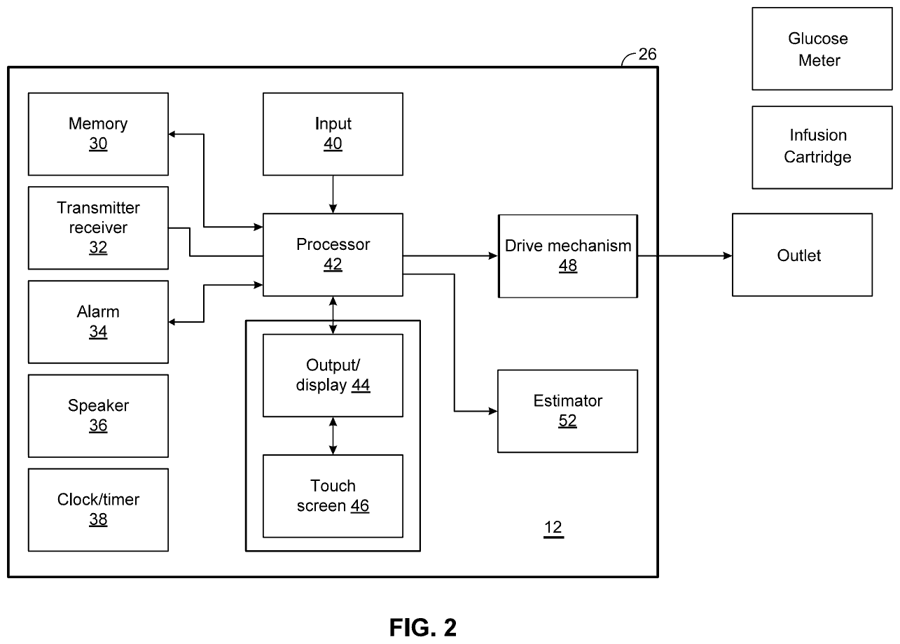

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices, for example, a CGM, display device, smartphone, etc. through the transmitter/receiver. The processor 42 may also include programming that may allow the processor to receive signals and/or other data from an input device, such as a sensor that may sense pressure, temperature, or other parameters.

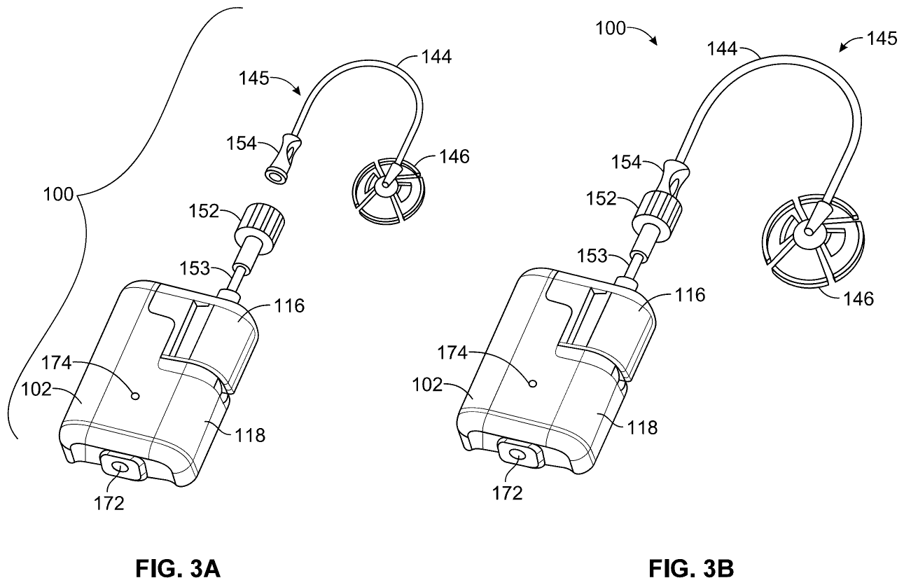

FIGS. 3A-3B depict another pump system including a pump 102 that can be used with embodiments. Drive unit 118 of pump 102 includes a drive mechanism 12 that mates with a recess in disposable cartridge 116 of pump 102 to attach the cartridge 116 to the drive unit 118. Pump system 100 can further include an infusion set 145 having a connector 154 that connects to a connector 152 attached to pump 102 with tubing 153. Tubing 144 extends to a site connector 146 that can attach or be pre-connected to a cannula and/or infusion needle that punctures the patient's skin at the infusion site to deliver medicament from the pump 102 to the patient via infusion set 145. In some embodiments, pump can include a user input button 172 and an indicator light 174 to provide feedback to the user.

In one embodiment, pump 102 includes a processor that controls operations of the pump and, in some embodiments, may receive commands from a separate device for control of operations of the pump. Such a separate device can include, for example, a dedicated remote control or a smartphone or other consumer electronic device executing an application configured to enable the device to transmit operating commands to the processor of pump 102. In some embodiments, processor can also transmit information to one or more separate devices, such as information pertaining to device parameters, alarms, reminders, pump status, etc. In one embodiment, pump 102 does not include a display but may include one or more indicator lights 174 and/or one or more input buttons 172. Pump 102 can also incorporate any or all of the features described with respect to pump 12 in FIG. 1. Further details regarding such pumps can be found in U.S. Pat. No. 10,279,106 and U.S. Patent Publication Nos. 2016/0339172 and 2017/0049957, each of which is hereby incorporated herein by reference in its entirety.

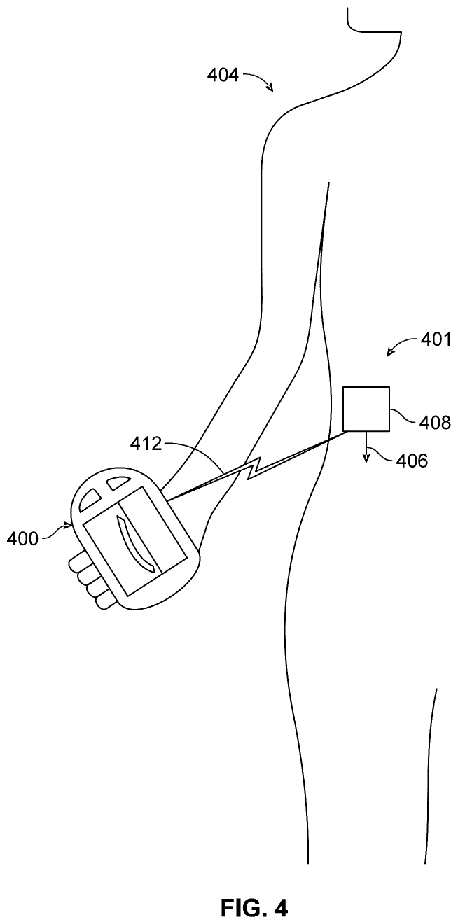

In some embodiments, the pump 12 or 102 can interface directly or indirectly (via, e.g., a smartphone or other device) with a glucose meter, such as a blood glucose meter (BGM) or a CGM. Referring to FIG. 4, an exemplary CGM system 400 according to an embodiment of the present invention is shown (other CGM systems can be used). The illustrated CGM system includes a sensor 401 affixed to a patient 404 that can be associated with the insulin infusion device in a CGM-pump system. The sensor 401 includes a sensor probe 406 configured to be inserted to a point below the dermal layer (skin) of the patient 404. The sensor probe 406 is therefore exposed to the patient's interstitial fluid or plasma beneath the skin and reacts with that interstitial fluid to produce a signal that can be associated with the patient's blood glucose level. The sensor 401 includes a sensor body 408 that transmits data associated with the interstitial fluid to which the sensor probe 406 is exposed. The data may be transmitted from the sensor 401 to the glucose monitoring system receiver 400 via a wireless transmitter, such as a near field communication (NFC) radio frequency (RF) transmitter or a transmitter operating according to a “Wi-Fi” or Bluetooth® protocol, Bluetooth® low energy protocol or the like, or the data may be transmitted via a wire connector from the sensor 401 to the monitoring system 400. Transmission of sensor data to the glucose monitoring system receiver by wireless or wired connection is represented in FIG. 4 by the arrow line 412. Further detail regarding such systems and definitions of related terms can be found in, e.g., U.S. Pat. Nos. 8,311,749, 7,711,402 and 7,497,827, as well as PCT Patent App. No. PCT/US23/82084, each of which is hereby incorporated by reference in its entirety.

In an embodiment of a pump-CGM system having a pump 12, 102 that communicates with a CGM and that integrates CGM data and pump data as described herein, the CGM can automatically transmit the glucose data to the pump. The pump can then automatically determine therapy parameters and deliver medicament based on the data. Such an automatic pump-CGM system for insulin delivery can be referred to as an automated insulin delivery (AID) or an artificial pancreas system that provides closed-loop therapy to the patient to approximate or even mimic the natural functions of a healthy pancreas. In such a system, insulin doses are calculated based on the CGM readings (that may or may not be automatically transmitted to the pump) and are automatically delivered to the patient at least in part based on the CGM reading(s). In various embodiments, doses can be delivered as automated correction boluses and/or automated increases or decreases to a basal rate. Insulin doses can also be administered based on current glucose levels and/or predicted future glucoses levels based on current and past glucose levels.

For example, if the CGM indicates that the user has a high blood glucose level or hyperglycemia, the system can automatically calculate an insulin dose necessary to reduce the user's blood glucose level below a threshold level or to a target level and automatically deliver the dose. If the CGM data indicates that the user has a low blood glucose level or hypoglycemia, the system can, for example, automatically reduce a basal rate and/or make other suggestions as may be appropriate to address the hypoglycemic condition. As with other parameters related to therapy, such thresholds and target values can be stored in memory located in the pump and the pump processor can periodically and/or continually execute instructions for a checking function that accesses these data in memory, compares them with data received from the CGM and acts accordingly to adjust therapy. The complexity of the algorithm used to calculate the insulin doses is therefore limited by the capabilities of the pump processor, memory, battery, etc.

In some embodiments, systems include an infusion pump, which can receive dosing functions from an electronic device or a neural network, select one of the dosing functions based on characteristics of a user, deliver a dose of medicament to the user according to the selected dosing function. The characteristics may include a basal rate, a correction factor, or a matching factor estimated based on the basal rate and the correction factor. Further detail regarding such systems and definitions of related terms can be found in PCT Patent App. No. PCT/US23/82084, which is hereby incorporated by reference in its entirety.

In some embodiments, the electronic device may be a mobile device (e.g. a phone, a tablet, a computer, etc.) of the user. The mobile device typically has more computation power than the infusion pump. For example, a smartphone can have 5 to 10 orders of magnitude more capability than an embedded device like an infusion pump. As such, various functions and algorithms for computing the insulin doses can be shifted from the pump processor to a processor at the mobile device. The infusion pump and the mobile device can be connected to each other via Internet, Wi-Fi, Bluetooth, near field communication (NFC), etc.

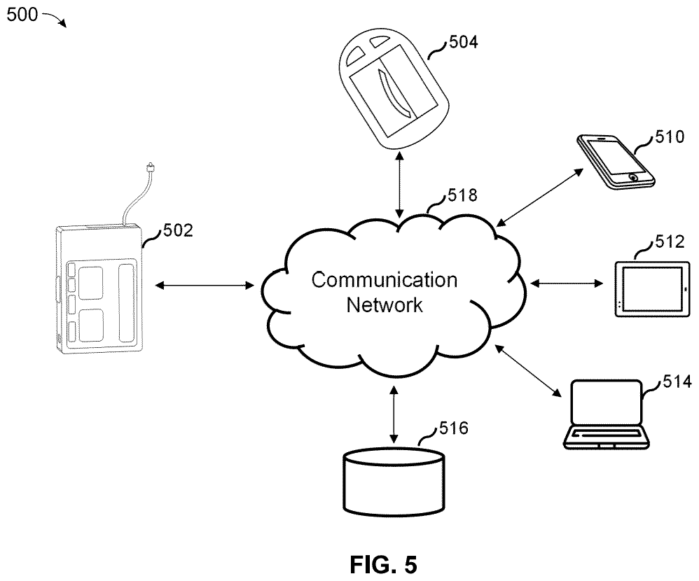

Embodiments disclosed herein employ a dynamic medicament infusion system that includes both a mobile device associated with a user and an infusion pump configured to deliver medicament doses to the user. FIG. 5 is a network environment 500 configured for automated medicament infusion, e.g. automated insulin delivery, according to various embodiments of the present disclosure.

As shown in FIG. 5, the network environment 500 includes a plurality of devices or systems configured to communicate over one or more network channels, illustrated as a network 518. For example, in various embodiments, the network environment 500 can include, but not limited to, a pump 502, a monitoring system 504 (e.g., a BGM system or a CGM system), a database 516, and one or more user computing devices 510, 512, 514 operatively coupled over the network 518. The pump 502, the monitoring system 504, and the multiple user computing devices 510, 512, 514 can each be any suitable computing device that includes any hardware or hardware and software combination for processing and handling information. For example, each can include one or more processors, one or more field-programmable gate arrays (FPGAs), one or more application-specific integrated circuits (ASICs), one or more state machines, digital circuitry, or any other suitable circuitry. In addition, each can transmit and receive data over the communication network 518.

In some examples, the pump 502 can be implemented as the pump 12 shown in FIG. 1 and FIG. 2, or as the pump 102 shown in FIG. 3. The monitoring system 504 may be implemented as the CGM system 400 shown in FIG. 4. In some examples, each of the multiple user computing devices 510, 512, 514 can be a cellular phone, a smart phone, a tablet, a personal assistant device, a voice assistant device, a digital assistant, a laptop, a computer, or any other suitable device. In some examples, each of the multiple user computing devices 510, 512, 514 includes one or more processing units, such as one or more graphical processing units (GPUs), one or more central processing units (CPUs), and/or one or more processing cores.

In some examples, the pump 502, the monitoring system 504, and the multiple user computing devices 510, 512, 514 are all associated with a same user. The monitoring system 504 is configured to monitor blood glucose level of the user, by continuously estimating the blood glucose level at each time step. The pump 502 is configured to deliver medicament, e.g. insulin, to the user based on the estimated blood glucose level from the monitoring system 504. The multiple user computing devices 510, 512, 514 are owned by or associated with the user, e.g. an account of the user has been logged in at these user computing devices 510, 512, 514. In addition, the same account of the user are also linked to or associated with the pump 502 and the monitoring system 504. In some embodiments, other methods (e.g. based on Bluetooth or NFC) can be used to pair the pump 502 and/or the monitoring system 504 to one or more of the multiple user computing devices 510, 512, 514. The pairing information may be saved securely at all these devices even when they are disconnected (e.g. due to power off or network interruption). As such, when they are reconnected via the network 518, they can be paired again automatically.

In some embodiments, to implement more powerful and complicated algorithms for medicament dose determination, and to avoid computational load of the algorithms at the pump 502, at least one of the multiple user computing devices 510, 512, 514 of the user works in cooperation with the pump 502 to compute optimal insulin doses at each time step for delivery to the user. For example, the user computing device 510 may be connected to the pump 502 via the network 518, and obtain dosing information associated with the user from the pump 502. The dosing information may include but not limited to: data from the monitoring system 504, an estimated glucose influx of the user, a dosing history of the pump 502 to the user, and information about a current pump state of the pump 502. The user computing device 510 can compute dosing models based on the dosing information. The dosing models may include both an online dosing model to be used by the pump 502 when the pump 502 is connected to the user computing device 510, and an offline dosing model to be used by the pump 502 when the pump 502 is not connected to the user computing device 510. The pump 502 will implement the online dosing model or the offline dosing model obtained from the mobile device, based on a connection status between the pump 502 and the user computing device 510.

In some embodiments, the offline dosing model includes a plurality of dosing functions corresponding to a plurality of predicted pump states respectively. Each of the dosing functions represents a corresponding dosing target associated with a corresponding predicted pump state. As such, even if the pump 502 is not connected to the user computing device 510, so long as a current pump state of the pump 502 falls into one of the plurality of predicted pump states, a corresponding pre-computed dosing function in the offline dosing model can be used by the pump 502 to determine and deliver a dose of medicament (e.g. insulin) to the user.

Although FIG. 1 illustrates three user computing devices 510, 512, 514, the network environment 500 can include any number of user computing devices 510, 512, 514. Similarly, the network environment 500 can include any number of the pumps 502, the monitoring systems 504, and the databases 116. That is, multiple users' medicament infusion systems can be implemented via the network 518 in the network environment 500.

The communication network 518 can be a WiFi® network, a cellular network such as a 3GPP® network, a Bluetooth® network, a satellite network, a wireless local area network (LAN), Wi-Fi network, a network utilizing radio-frequency (RF) communication protocols, a Near Field Communication (NFC) network, a wireless Metropolitan Area Network (MAN) connecting multiple wireless LANs, a wide area network (WAN), or any other suitable network. The communication network 518 can provide access to, for example, the Internet.

In some embodiments, each of the first user computing device 510, the second user computing device 512, and the Nth user computing device 514 may communicate with the pump 502 over the communication network 518. For example, each of the multiple user computing devices 510, 512, 514 may establish a connection to the pump 502. In some embodiments, the pump 502 works with only one of the multiple user computing devices 510, 512, 514 at any given time, while the other user computing devices may work as a relay for forwarding signals back and forth. In some embodiments, the pump 502 works with multiple of the multiple user computing devices 510, 512, 514 at a given time. In some embodiments, the pump 502 switches working from one of the multiple user computing devices 510, 512, 514 to another, based on their corresponding connection statuses.

In some examples, the monitoring system 504 transmits to the pump 502 signals indicating a blood glucose level of the user at each time step. In some examples, the pump 502 may execute one or more models (e.g., programs or algorithms), to generate and deliver a dose of medicament (e.g. insulin) to the user. The models may be pre-computed by one of the user computing devices 510, 512, 514. In some examples, at least one of the models in a machine learning model, deep learning model, statistical model, etc.

In some embodiments, the pump 502 is further operable to communicate with the database 516 over the communication network 518. For example, the pump 502 can store data to, and read data from, the database 516. The database 516 can be a remote storage device, such as a cloud-based server, a disk (e.g., a hard disk), a memory device on another application server, a networked computer, or any other suitable remote storage. Although shown remote to the pump 502, in some examples, the database 516 can be a local storage device, such as a hard drive, a non-volatile memory, or a USB stick. For example, the pump 502 may store the online dosing model and/or the offline dosing model received from the user computing device 510 in the database 516. The pump 502 may receive measurement data from the monitoring system 504 and store them in the database 516. The pump 502 may also store previous dosing history data and/or pump state history data in the database 516.

In some examples, the user computing device 510 generates and/or updates different models (e.g., online dosing model, offline dosing model, open loop model, etc.) for medicament dose computation. When some models are machine learning models or deep learning models, the user computing device 510 may generate training data for the models based on data including but not limited to: historical dosing information of the pump 502, historical dosing data of the pump 502, historical blood glucose levels of the user, historical pump states of the pump 502. The user computing device 510 trains the models based on their corresponding training data, and store the models in a database, such as in the database 516 (e.g., a cloud storage or a local storage). The models, when executed by the pump 502, allow the pump 502 to determine and deliver doses of medicament to the user, with or without connection to the user computing device 510.

In some examples, the user computing device 510 assigns the models (or parts thereof) for execution to one or more processing devices (not shown) connected to the network 518. For example, each model may be assigned to a virtual machine hosted by a processing device. The virtual machine may cause the models or parts thereof to execute on one or more processing units such as GPUs. In some examples, the virtual machines assign each model (or part thereof) among a plurality of processing units. Based on the output of the models, the pump 502 may determine medicament infusion data.

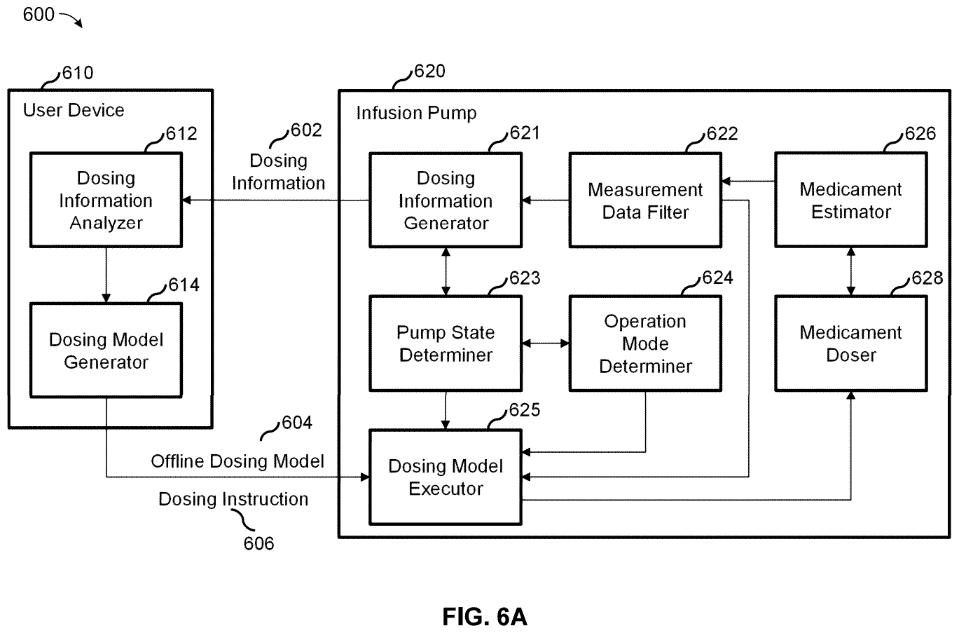

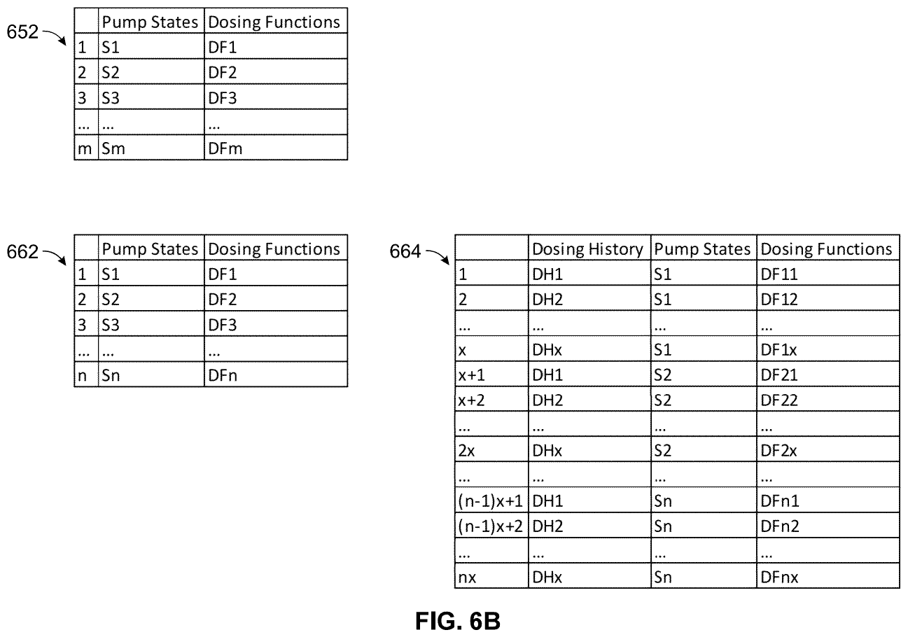

FIG. 6A is a block diagram of a system 600 for employing automated insulin delivery with a user device and an infusion pump, according to various embodiments of the present disclosure. Referring to FIG. 6A, the system 600 includes a user device 610 and an infusion pump 620. In some embodiments, an automated glycemic control algorithm is implemented across the user device 610 and the infusion pump 620 to provide optimized insulin delivery to a user.

In some embodiments, the user device 610 is a mobile device (e.g. a phone, tablet, watch, wearable device or laptop) associated with the user, and the infusion pump 620 is configured to deliver medicament doses to the user. In some examples, the user device 610 may be implemented as one of the user computing devices 510, 512, 514 shown in FIG. 5. The infusion pump 620 may be implemented as the pump 12 shown in FIG. 1 and FIG. 2, the pump 102 shown in FIG. 3, or the pump 502 shown in FIG. 5. In some embodiments, each of the user device 610 and the infusion pump 620 includes components connected in a scheme as shown in the housing 26 in FIG. 2.

As shown in FIG. 6A, the user device 610 includes a dosing information analyzer 612 and a dosing model generator 614. The infusion pump 620 in this example includes: a dosing information generator 621, a measurement data filter 622, a pump state determiner 623, an operation mode determiner 624, a dosing model executor 625, a medicament estimator 626, and a medicament doser 628. In some examples, one or more of the dosing information analyzer 612, the dosing model generator 614, the dosing information generator 621, the measurement data filter 622, the pump state determiner 623, the operation mode determiner 624, the dosing model executor 625, the medicament estimator 626 and the medicament doser 628 are implemented in hardware. In some examples, one or more of the dosing information analyzer 612, the dosing model generator 614, the dosing information generator 621, the measurement data filter 622, the pump state determiner 623, the operation mode determiner 624, the dosing model executor 625, the medicament estimator 626 and the medicament doser 628 are implemented as an executable program maintained in a tangible, non-transitory memory, such as the memory 30 in FIG. 2, which may be executed by one or more processors, such as the processor 42 in FIG. 2.

The dosing information analyzer 612 in this example can receive dosing information 602 associated with the user from the dosing information generator 621. The dosing information 602 may comprise at least one of: data from a CGM associated with the user, an estimated glucose influx of the user based on the data from the CGM, a dosing history of the infusion pump 620 regarding the user, or information about a current pump state of the infusion pump 620. The dosing information analyzer 612 many analyze the dosing information 602 by extracting data from the dosing information 602, and forward the data to the dosing model generator 614 for dosing model generation.

The dosing model generator 614 in this example obtains the data extracted from the dosing information 602, and determines both an online dosing model and an offline dosing model. In some embodiments, the dosing model generator 614 may generate the online dosing model for dosing determination based on all possible pump states and user states, in view of historical pump data and user data from a large user pool. In some embodiments, the dosing model generator 614 may obtain the online dosing model from a cloud server connected to a plurality of users and a plurality of pumps. The online dosing model may be updated by the dosing model generator 614 or the cloud server at predetermined time periods. In some examples, the dosing model generator 614 may generate a dosing instruction 606 for the user based on the online dosing model and the dosing information 602, and transmit the dosing instruction 606 to the infusion pump 620 at periodic time intervals, e.g. every minute or every 5 minutes. In some embodiments, the dosing model generator 614 transmits the dosing instruction 606 to the infusion pump 620 in real time, once the dosing instruction 606 is generated.

In some examples, the dosing model generator 614 may determine a plurality of predicted pump states in a predetermined time period (e.g. in the next hour or next 5 hours) based on the dosing information 602 associated with the user, and generate an offline dosing model 604 for the user in the predetermined time period based on the online dosing model and the plurality of predicted pump states. The offline dosing model 604 may have a smaller size than the online dosing model. In some embodiments, the dosing model generator 614 updates and transmits the offline dosing model 604 to the infusion pump 620, e.g. the dosing model executor 625 in the infusion pump 620, at periodic time intervals, e.g. every hour or every 5 hours.

In some embodiments, the offline dosing model 604 comprises a look-up table indicating the plurality of predicted pump states and a plurality of dosing functions corresponding to the plurality of predicted pump states respectively. Each of the dosing functions may represent a corresponding dosing target associated with a corresponding predicted pump state of the infusion pump 620.

In the infusion pump 620, the dosing information generator 621 is configured to generate the dosing information associated with the user, e.g. based on filtered measurement data from the measurement data filter 622 and a pump state determined by the pump state determiner 623. The measurement data filter 622 in this example may filter measurement data of the user from a monitor, e.g., a CGM. In some embodiments, an estimated value is computed based on applying a filter on the measurement result on the user from the monitor. In some examples, the filter may be a Kalman filter, and can be updated based on a dose of medicament delivered to the user at a previous time step. In some embodiments, the estimated value represents an estimated glucose influx for the user at a current time step.

The pump state determiner 623 in this example can determine a pump state of the infusion pump 620 at any given time step. In some embodiments, the pump state of the infusion pump 620 is determined based on the dosing information (including information about previous pump states). In some embodiments, the pump state of the infusion pump 620 is also determined based on one or more characteristics of the user. For example, the one or more user characteristics comprise: a target blood glucose, a basal rate, a correction factor, and/or a carb ratio of the user. The one or more characteristics of the user may be obtained by the dosing information generator 621 or by the pump state determiner 623 itself, e.g. based on previous measurement or user inputs before running the algorithms at the user device 610 and the infusion pump 620. In some embodiments, the pump state determiner 623 may determine a pump state of the infusion pump 620 at each time step based on the dosing information and the one or more characteristics of the user. In some embodiments, the offline dosing model 604 may be customized to the user based on the one or more characteristics of the user. In some embodiments, a same online dosing model and/or a same offline dosing model may be generated to fit many different users.

In some embodiments, the system computes an insulin dosing target for the user based on not only the data from the CGM, but also an activity state of the user. Activity states may include meal states, exercise states, or both. For example, while the user is not eating, the not eating state may be further divided into different exercise states: e.g., not exercising, exercising start, exercising heavily, exercising slightly, exercising finish. For example, given a same measurement result from the CGM indicating a same blood glucose level, the system can compute a first insulin dosing target if the user just starts to cat (at a first state), and compute a second insulin dosing target if the user is about to finish eating (at a second state). In some examples, the first insulin dosing target may be higher than the second insulin dosing target. This is because the user at the first state will receive more glucose soon, but the user at the second state will stop receiving glucose soon. In addition, it takes some time for dosed insulin to take effect in the user's body. Accordingly, the pump state of the infusion pump 620 may be determined based on determining a plurality of activity states. The user is in one of the activity states at any given time and capable of transitioning among the activity states from time to time. Then, the infusion pump 620 determines state scores each representing a probability that the user is in a respective one of the activity states at the given time step, e.g., based on a hidden Markov model (HMM), and determines the pump state of the infusion pump 620 based on: the dosing information, the one or more characteristics of the user, and the state scores.

The operation mode determiner 624 in this example can determine an operation mode of the infusion pump 620 at a given time step, based on a connection status between the user device 610 and the infusion pump 620. In some embodiments, the operation mode comprises an online mode when the infusion pump 620 is connected to the user device 610 and an offline mode when the infusion pump 620 is not connected to the user device 610. In some embodiments, a connection between the user device 610 and the infusion pump 620 is based on at least one of: Internet, Wi-Fi, Bluetooth, or near field communication (NFC). The operation mode determiner 624 may perform a check of the connection status between the user device 610 and the infusion pump 620 at periodic time intervals, e.g. every minute, every 5 minutes or every 10 minutes.

The dosing model executor 625 in this example can obtain the dosing instruction 606 from the dosing model generator 614 in real time, and obtain the offline dosing model 604 from the dosing model generator 614 at periodic time intervals. In some examples, the dosing model executor 625 may store the offline dosing model in a local storage of the infusion pump 620.

In some examples, in accordance with a determination by the operation mode determiner 624 that the infusion pump 620 is in an online mode, the dosing model executor 625 may determine a dose of medicament to be delivered to the user based on the dosing instruction 606, and then inform the medicament doser 628 about the dose of medicament for automated delivery. In some examples, in accordance with a determination by the operation mode determiner 624 that the infusion pump 620 is in an offline mode, the dosing model executor 625 executes the offline dosing model 604 to determine a dose of medicament to be delivered to the user, and then informs the medicament doser 628 about the dose of medicament for automated delivery.

In some embodiments, after the operation mode determiner 624 determines that the infusion pump 620 is in the offline mode, the operation mode determiner 624 asks the pump state determiner 623 to determine a current pump state of the infusion pump 620. After receiving the current pump state from the pump state determiner 623, the dosing model executor 625 can determine a predicted pump state of the offline dosing model that corresponds to the current pump state, and determine the dose of medicament based on the offline dosing model 604 and a respective dosing function corresponding to the predicted pump state in the offline dosing model.

In some embodiments, the offline dosing model 604 comprises a look-up table, e.g. the look-up table 652 as shown in FIG. 6B, indicating the plurality of dosing functions and the corresponding plurality of predicted pump states. Accordingly, the dosing model executor 625 can execute the offline dosing model 604 in the offline mode by: selecting, from the look-up table, the predicted pump state that is closest to the current pump state among the plurality of predicted pump states; identifying, from the plurality of dosing functions, a dosing function corresponding to the predicted pump state; and determining the dose of medicament in the offline mode based on the dosing function. As such, the look-up table in the offline dosing model can provide a pre-computed decision tree for the infusion pump 620 to easily determine an optimal dose even in offline mode. The look-up table 652 in this example includes m pump states and m corresponding dosing functions.

In some embodiments, the online dosing model may be stored in the user device 610 or a cloud server connected to the user device 610. The online dosing model may indicate a dosing target for the user based on the dosing information 602. The dosing instruction may include the dosing target when the infusion pump 620 is in the online mode. In this case, the dosing model executor 625 in the online mode can directly forward the dosing target to the medicament doser 628 for delivery or compute a dose of medicament based on the dosing target for delivery at the medicament doser 628, without a need of the pump state of the infusion pump 620 anymore.

In some embodiments, the online dosing model indicates dosing functions corresponding to a set of pump states of the infusion pump of the infusion pump 620, e.g. using a look-up table. For example, the look-up table 662 as shown in FIG. 6B may be used as an online dosing model. The look-up table 662 in this example includes n pump states and n corresponding dosing functions. In some embodiments, the m pump states in the look-up table 652 are selected from the n pump states in the look-up table 662 to represent m most likely pump states in a future time period; the m dosing functions in the look-up table 652 are selected from the n dosing functions in the look-up table 662 to represent dosing functions corresponding to the m most likely pump states in the future time period.

In some embodiments, the online dosing model indicates dosing functions corresponding to a plurality of pump states of the infusion pump 620 based on dosing history data. For example, the look-up table 664 as shown in FIG. 6B may be used as an online dosing model based on both dosing history and pump states. The look-up table 664 in this example includes n pump states and x dosing history states for each of the n pump states. A dosing function may be determined for each pair of dosing history and pump state. In some embodiments, the offline dosing model may also include a similar look-up table based on both dosing history and pump states.

For example, when the user device 610 is connected to the infusion pump 620, the user device 610 continuously accumulates the dosing history of the infusion pump 620, and computes, based on a current pump state and all the dosing history of the infusion pump 620, dosing targets or functions for many or all different potential future pump states of the infusion pump 620. The online dosing model may include dosing results for all of the different potential future pump states.

In some embodiments, while the plurality of predicted pump states of the offline dosing model correspond to a first number of pump states of the infusion pump 620, the pump states of the online dosing model correspond to a second number of pump states of the infusion pump 620. In some examples, the second number is larger than or equal to the first number. In some examples, the first number of pump states are selected from the second number of pump states to represent most likely pump states in a future time period. In some embodiments, for a same pump state in both the offline dosing model and the online dosing model, a same dosing function corresponding to the pump state is used in both the offline dosing model and the online dosing model. In some embodiments, for a same pump state in both the offline dosing model and the online dosing model, the corresponding dosing function in the offline dosing model is a quantized version of the corresponding dosing function in the online dosing model.

In some examples, the user device 610 can update the offline dosing model 604 at periodic time intervals or upon a triggering event. Each offline dosing model generated or updated by the user device 610 may have a size smaller than a size limit associated with the local storage of the infusion pump 620. The user device 610 may transmit a most updated version of the offline dosing model 604 to the infusion pump 620 when the infusion pump 620 is connected to the user device 610.

In some embodiments, the user device 610 may update the offline dosing model 604 based at least in part by: determining an updated time period based on a triggering event or a user input of the user, determining a plurality of updated pump states in the updated time period based on the dosing information 602 associated with the user, and generating an updated offline dosing model 604 for the user in the updated time period based on the online dosing model 604 and the plurality of updated pump states. In some embodiments, the updated time period is longer than the predetermined time period associated with the original offline dosing model. For example, while the original offline dosing model was generated for one hour, the offline dosing model needs to be updated for using in two or more hours. The updated offline dosing model may have a lower fidelity level than the offline dosing model with respect to the online dosing model. That is, there may be a tradeoff between fidelity and durability for an offline dosing model due to a size limit of the offline dosing model.

In some embodiments, the user device 610 may update the offline dosing model 604 based at least in part by: determining that the infusion pump 620 switches from the offline mode to the online mode, determining a last version of the offline dosing model 604 used by the infusion pump 620 during the offline mode, comparing the online dosing model with the last version of the offline dosing model 604 based on the dose of medicament delivered during the offline mode to generate a comparison result, and generating an updated offline dosing model 604 for the user in a next time period based on the online dosing model and the comparison result. The offline dosing model 604 may be updated or recalibrated based on a feedback loop from the infusion pump 620 to the user device 610.

In some examples, the user device 610 can predict the most likely pump states in the next time period or a future time period for the infusion pump 620, e.g. based on the dosing information 602, and generate or update the offline dosing model 604 merely based on these most likely pump states, e.g. pump states having a probability larger than a threshold to happen in the future time period for the infusion pump 620. The offline dosing model 604 can be generated without knowing whether and when the infusion pump 620 and the user device 610 are disconnected from each other in the future time period (e.g. next hour or next day). But since the offline dosing model 604 is updated periodically, e.g. every 10 minutes or every hour, once the infusion pump 620 and the user device 610 are disconnected from each other, the most updated offline dosing model can be used by the infusion pump 620 to handle these most likely pump states in the offline mode. Every time the user device 610 is reconnected to the infusion pump 620, the user device 610 computes a new offline dosing model that the infusion pump 620 is capable of running later. As such, the computational load is shifted from the infusion pump 620 to the user device 610 without highly coupling the connectivity of the two devices together. The user does not need to worry about the connectivity of the two devices either, as the offline dosing model can be automatically run by the infusion pump 620 in the offline mode. As such, the offline mode is a closed loop mode where no user input or operation is needed for dose delivery. In some embodiments, the infusion pump 620 may execute the offline dosing model even in the online mode, to save operation time and power.

In some embodiments, the operation mode of the infusion pump 620 further comprises an open loop mode when: (a) the infusion pump 620 is not connected to the user device 610 and the current pump state of the infusion pump 620 is unknown or outside the plurality of predicted pump states in the predetermined time period, or (b) the infusion pump 620 is still not connected to the user device 610 after the predetermined time period. In some examples, when the connection status between the user device 610 and the infusion pump 620 is worse than a threshold, and when the pump state of the infusion pump 620 is determined by the pump state determiner 623 to be not matching any of the predicted pump states in the offline dosing model 604, the infusion pump 620 operates in the open loop mode. In accordance with a determination by the operation mode determiner 624 that the infusion pump is in an open loop mode, the dosing model executor 625 executes an open loop model based on a predetermined profile of the user and/or manual operation of the user, to determine a dose of medicament to be delivered to the user. In some embodiments, in accordance with a determination by the operation mode determiner 624 that the infusion pump is in an open loop mode, the dosing model executor 625 may determine a fixed and predetermined dose of medicament to be delivered to the user based on a predetermined profile of the user.

In some examples, the infusion pump 620 switches from the offline mode to the open loop mode in accordance with a determination that the CGM associated with the user is disconnected from the infusion pump 620. In some examples, the infusion pump 620 switches from the open loop mode to the offline mode in accordance with a determination that the CGM is reconnected to the infusion pump 620.

In some embodiments, at least one of the user device 610 or the infusion pump 620 is configured to provide to the user, via a user interface, a first notification that the user device 610 is disconnected from the infusion pump 620, when the connection status is worse than a threshold. In some embodiments, the infusion pump 620 is configured to provide to the user, via a user interface, a second notification that the infusion pump 620 is in the open loop mode, when the infusion pump enters the open loop mode. The first notification and the second notification may include: a message on a screen and/or a vibration in a particular pattern.

The medicament doser 628 in this example is configured to determine an optimal amount of insulin dose and deliver the optimal amount of insulin dose to the user, based on the model execution results of the dosing model executor 625. In some embodiments, the medicament doser 628 can estimate an unmetabolized insulin (UMI), approximate insulin onboard (IOB), and generate glucose predictions. In some embodiments, the medicament doser 628 considers all insulin active in the body of the user based on insulin estimate by the medicament estimator 626. In some embodiments, the dosed amount at each time step by the medicament doser 628 is provided to the medicament estimator 626 as feedback.

The medicament estimator 626 in this example can estimate a current insulin amount (or in general an amount of medicament being dosed) in the body of the user based on: previous doses performed by the medicament doser 628. In some examples, the medicament doser 628 can compute the optimal amount of insulin dose based on: the estimated current insulin amount from the medicament estimator 626 and the optimum insulin dosing target from the dosing model executor 625, for the current time step. In some embodiments, an estimated IOB mean is computed by the medicament estimator 626 and provided to the measurement data filter 622 for filtering the estimated glucose influx.

In some embodiments, one or more of the components in the infusion pump 620 can be further shifted to the user device 610. For example, the medicament estimator 626 can be shifted from the infusion pump 620 to the user device 610. The function of the measurement data filter 622 can be shifted from the infusion pump 620 to the user device 610 in the online mode, and shifted back from the user device 610 to the infusion pump 620 in the offline mode. In some embodiments, all computations for determining the optimal insulin dose are performed by the user device 610. In some embodiments, only computations having a complexity or intensity higher than a threshold for determining the optimal insulin dose are performed by the user device 610.

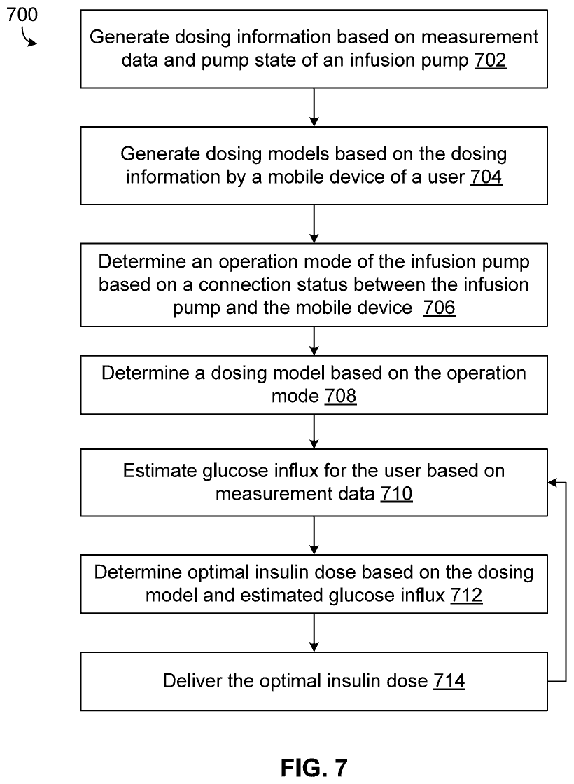

Referring now to FIG. 7, a flow chart of a method 700 for delivering optimal insulin doses to a user is depicted, according to various embodiments of the present disclosure. In some embodiments, the method 700 can be implemented through the system 600.

At step 702, dosing information is generated based on measurement data and pump state of an infusion pump, e.g. the infusion pump 620. In some embodiments, the step 702 is performed by the dosing information generator 621 in the infusion pump 620. The measurement data is obtained by the measurement data filter 622 from a CGM or a BGM associated with the user.

Dosing models are computed at step 704, based on the dosing information by a mobile device of the user, e.g. by the user device 610. In some embodiments, the step 704 is performed by the dosing model generator 614 in the user device 610. The dosing models may include an online dosing model and an offline dosing model.

At step 706, an operation mode of the infusion pump is determined based on a connection status between the infusion pump and the mobile device, e.g. between the infusion pump 620 and the user device 610. In some embodiments, the step 706 is performed by the operation mode determiner 624 in the infusion pump 620. The operation mode may comprise an online mode when the infusion pump is connected to the mobile device and an offline mode when the infusion pump is not connected to the mobile device.

A dosing model is then determined at step 708, based on the operation mode. In some embodiments, the step 708 is performed by the dosing model executor 625 in the infusion pump 620. In some examples, the dosing model is determined to be the online dosing model when the operation mode is the online mode, and determined to be the offline dosing model when the operation mode is the offline mode. As discussed above, in some embodiments, when the pump state of the infusion pump 620 is unknown (e.g. due to a disconnection from a CGM) or when the pump state is outside the predicted pump states in the offline dosing model (such that the memory runs out at the infusion pump 620), the infusion pump 620 operates in an open loop mode, where an open loop model is determined and executed.

At step 710, an estimated value (e.g., an estimated glucose influx) for a user is computed based on measurement data from a sensor, e.g., a CGM, for each dosing interval (e.g., every five minutes), which may be performed by the measurement data filter 622 in the infusion pump 620. In some embodiments, the measurement data is filtered to generate a distribution of glucose influx at a given time step. In that case, a mean of the distribution may be used as the estimated glucose influx. At step 712, appropriate doses (e.g., an optimal insulin dose) for each dosing interval (e.g., every five minutes) are determined based on the estimated glucose influx and the determined dosing model, which may be performed by the dosing model executor 625 in the infusion pump 620. Finally, at step 714, the insulin pump delivers the determined insulin dose, which may be performed by the medicament doser 628 in the infusion pump 620.

In some embodiments, steps 710 through 714 are performed for each dosing interval or time step and can be repeated as necessary to enable the automated glycemic control algorithm to operate ad infinitum without running out of policies. For example, at a next time step, the infusion pump can: (1) determine a new pump state of the infusion pump; (2) select a new dosing policy or function corresponding to the new pump state; and (3) deliver a new insulin dose to the user according to the new dosing policy, based on an updated estimate of the glucose influx of the user.

It should be understood that the individual operations used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

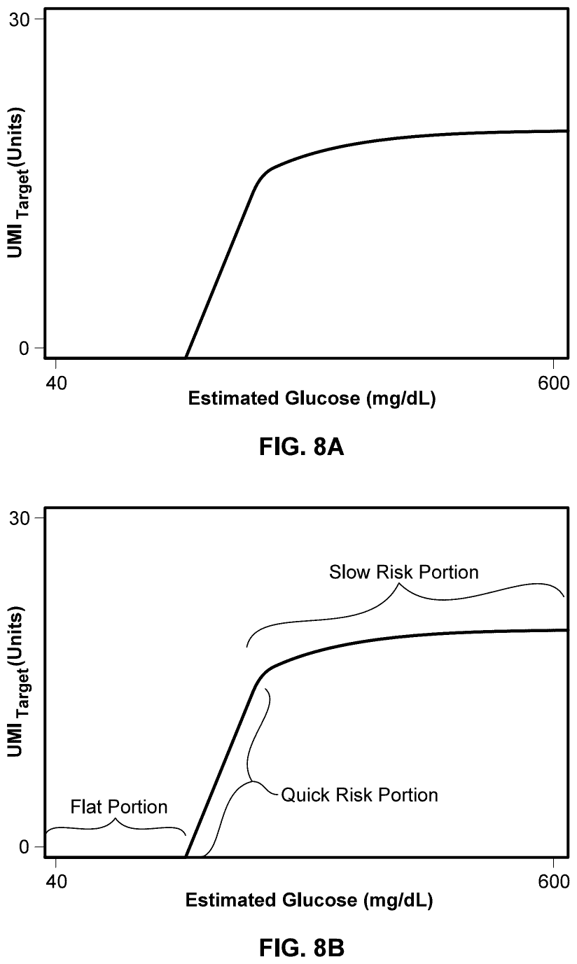

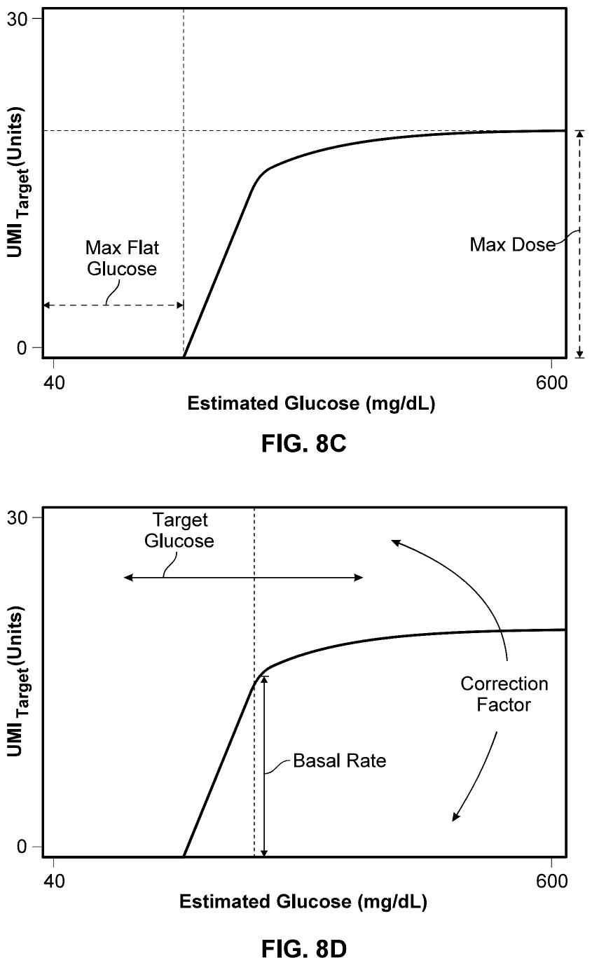

In some embodiments, dosing policies are lookup tables generated by an automated glycemic control algorithm policy generator. A typical policy (given an activity state of a user) is depicted in FIG. 8A according to an embodiment and elements of that policy are identified in FIGS. 8B-8D. Each policy defines the relationship between the estimated glucose and the UMI-Target over a range. In the embodiment depicted in FIGS. 8A-8D, the policy defines the range of 40 mg/dL to 600 mg/dL.

Referring to FIG. 8B, each policy includes three segments: a flat portion, a quick rise portion, and a slow rise portion. In the flat portion, UMI-Target should be zero as this represents flat glucose. In the quick rise portion, UMI-Target should rise approximately linearly (sometimes following a sigmoid) with predicted glucose, and the largest y-value should occur at the predicted glucose target. In the slow rise portion, UMI-Target shall have a positive first derivative and a negative second derivative. Each policy has both (i) a maximum dose and (ii) end of flat glucose value, as depicted in FIG. 8C. The impact of adjusting automated glycemic control algorithm parameters is illustrated in FIG. 8D. Adjusting automated glycemic control algorithm Target Glucose tends to translate the policy to the right or left. Adjusting automated glycemic control algorithm Basal tends to scale the policy up and down. Adjusting automated glycemic control algorithm Correction Factor tends to rotate the policy Clockwise (CW) or Counter-Clockwise (CCW).

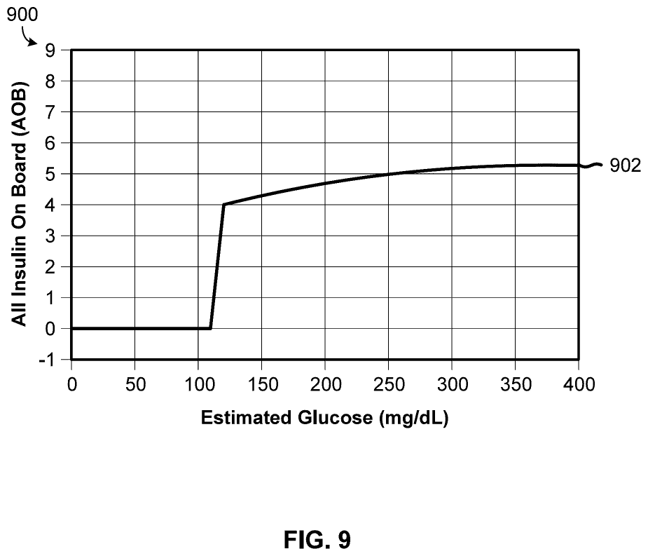

FIG. 9 illustrates optimal all-IOB (“aIOB” or sometimes referred to as “AOB”) target predictions 902, according to various embodiments of the present disclosure. The graph 900 illustrates a dosing function, with an aIOB target 902 as a function of estimated glucose. For example, a system (e.g., the system 600) can determine an optimal aIOB for a patient based in part on an estimated blood glucose level of the patient, given an activity state.

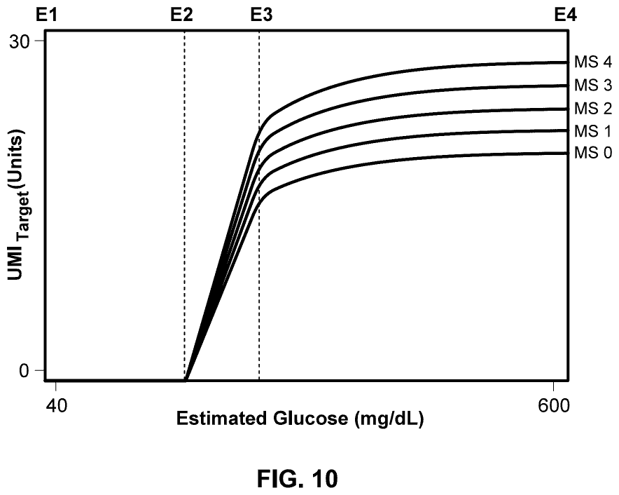

Because each policy can be personalized, each user will have an optimum policy given an activity state. FIG. 10 is a graph showing different policies corresponding to different activity states, according to various embodiments of the present disclosure. In the example shown in FIG. 10, dosing policies for five meal states (MS0, MS1, MS2, MS3, MS4) are illustrated. As shown in FIG. 10, the five dosing policies share the same flat portion between E2 and E2, have different quick rise portions between E2 and E3, and have different slow rise portions between E3 and E4. In some embodiments, the slopes of the quick rise portions of the five dosing policies can be blended based on a weighted average of these slopes, with weights being the corresponding state probabilities computed for the five meal states at a given time step and given an estimated glucose influx, to generate a blended slope. In some embodiments, each slow rise portion is modelled or fit by a same-order polynomial function with coefficients. Those slow rise portions are blended based on a weighted average of these coefficients, with weights being the corresponding state probabilities computed for the five meal states at a given time step and given an estimated glucose influx, to generate a set of blended coefficients. For example, each slow rise portion can be modelled as a fifth-order polynomial function with six coefficients. A weighted average of the six coefficients of all dosing policies can be computed to generate a set of blended six coefficients. The blended policy is then represented by: the same flat portion between E1 and E2, a quick rise portion with the blended slope between E2 and E3, and a slow rise portion with the blended coefficients between E3 and E4. In other embodiments, the different policies or functions in FIG. 10 can also represent an aIOB target as in FIG. 9, rather than representing UMI-Target as in FIG. 10. In some embodiments, the activity states of the user and their corresponding state probabilities are taken into consideration when generating the dosing information 602 and/or determining the pump state of the infusion pump 620.

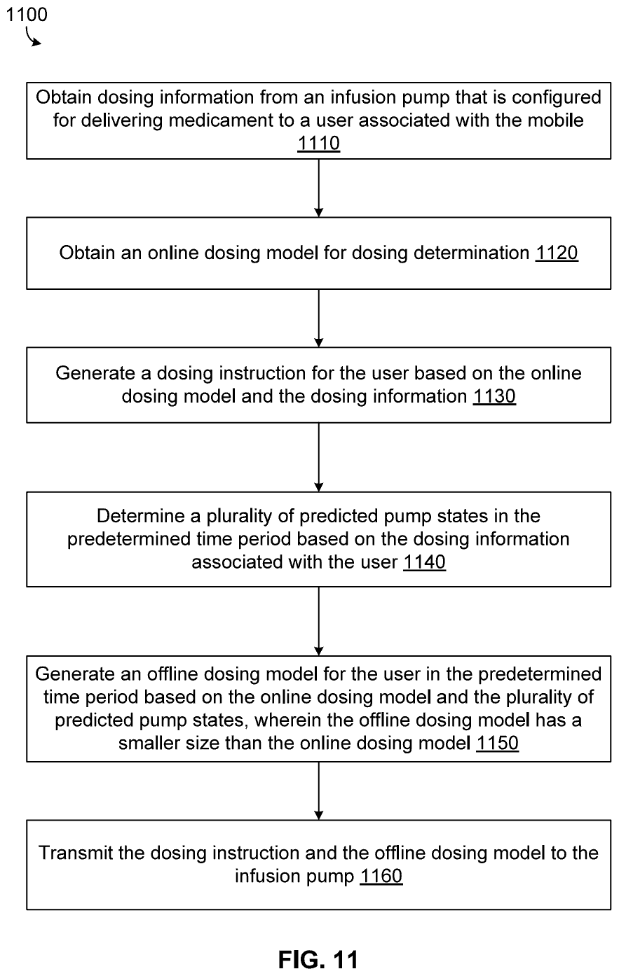

FIG. 11 is a flow chart of a method 1100 performed by a mobile device for automatically delivering a dose of medicament, according to various embodiments of the present disclosure. In some embodiments, the method 1100 is performed by the user device 610 as discussed above with respect to FIG. 6A. It is noted that some of the operations of the method 1100 can be performed in an order other than the serial order suggested by the FIG. 11 and the following discussion. Likewise, some of the operations can be performed in parallel, and, in some embodiments, some of the operations need not be performed whatsoever.

In the illustrated embodiment, the mobile device obtains (1110) dosing information from an infusion pump, e.g. the infusion pump 620, that is configured for delivering medicament to a user associated with the mobile device. The dosing information may be generated by the infusion pump and may include information related to the infusion pump and/or the user. For example, the dosing information may indicate, in a predetermined time period, whether the infusion pump is in an online mode when the infusion pump is connected to the mobile device or in an offline mode when the infusion pump is not connected to the mobile device. In some examples, the dosing information may be obtained by the dosing information analyzer 612 in FIG. 6A.

The mobile device may obtain (1120) an online dosing model for dosing determination. For example, the online dosing model may be obtained by the dosing model generator 614 in FIG. 6A. As discussed above, the online dosing model may be generated by the dosing model generator 614 or a cloud server connected to the mobile device.

After receiving the dosing information, the mobile device generates (1130) a dosing instruction for the user based on the online dosing model and the dosing information. For example, the dosing instruction may be generated by the dosing model generator 614 in FIG. 6A.

Based on the dosing information, the mobile device may also determine (1140) a plurality of predicted pump states in the predetermined time period, and generate (1150) an offline dosing model for the user in the predetermined time period based on the online dosing model and the plurality of predicted pump states. The offline dosing model may have a smaller size than the online dosing model. For example, the offline dosing model may be generated by the dosing model generator 614 in FIG. 6A.

Then the mobile device transmits (1160) the dosing instruction and the offline dosing model to the infusion pump. For example, the dosing instruction and the offline dosing model may be transmitted by the dosing model generator 614 in FIG. 6A. In some embodiments, the mobile device transmits the offline dosing model to the infusion pump, based on a request (standalone or embedded in the dosing information) from the infusion pump, where the request is generated based on an operation mode of the infusion pump.

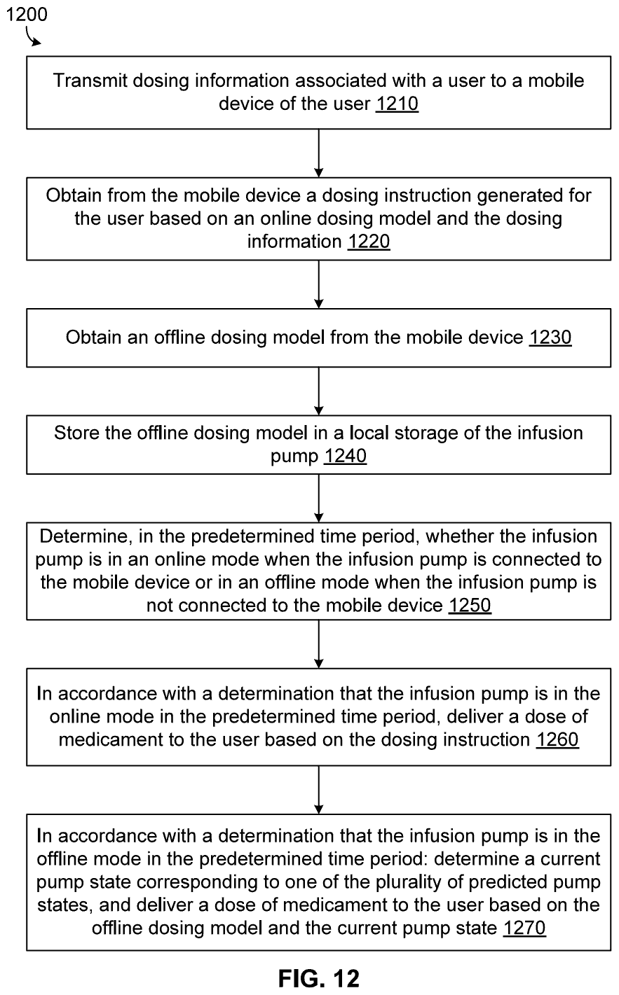

FIG. 12 is a flow chart of a method 1200 performed by an infusion pump for automatically delivering a dose of medicament, according to various embodiments of the present disclosure. In some embodiments, the method 1200 is performed by an infusion pump, e.g. the pump 12 shown in FIG. 1 and FIG. 2, the pump 102 shown in FIG. 3, the pump 502 shown in FIG. 5, or the infusion pump 620 shown in FIG. 6A.

For illustrative purposes, the following discussion refers to “a processor” as performing the operations of the method 1200. This processor may be a processor of the aforenoted infusion pump. Additionally, the processor may be a processor of a server, a mobile device, or any other electronic device discussed herein or otherwise known in the art. Moreover, it is noted that some of the operations of the method 1200 can be performed in an order other than the serial order suggested by the FIG. 12 and the following discussion. Likewise, some of the operations can be performed in parallel, and, in some embodiments, some of the operations need not be performed whatsoever.

In the illustrated embodiment, the processor transmits (1210) dosing information associated with a user to a mobile device (e.g. the user device 610) of the user. The dosing information may include but not limited to: data from a CGM or BGM, an estimated glucose influx of the user, a dosing history of the pump to the user, and information about a current pump state of the pump. In some embodiments, the dosing information may also include prediction data and weights related to different data in the dosing information. For example, the dosing information may be generated and transmitted by the dosing information generator 621 in FIG. 6A.

Then, the processor obtains (1220) from the mobile device (e.g. the user device 610) a dosing instruction generated for the user based on an online dosing model and the dosing information, and obtains (1230) an offline dosing model from the mobile device (e.g. the user device 610). For example, the dosing instruction and the offline dosing model may be obtained by the dosing model executor 625 in FIG. 6A.

In some embodiments, the offline dosing model is generated for the user in a predetermined time period based on the online dosing model and a plurality of predicted pump states. The plurality of predicted pump states may be determined for the predetermined time period based on the dosing information associated with the user. The offline dosing model may have a smaller size than the online dosing model.

The processor may store (1240) the offline dosing model in a local storage of the infusion pump. For example, the offline dosing model may be stored by the dosing model generator 614 in FIG. 6A.

In the predetermined time period, the processor may determine (1250) whether the infusion pump is in an online mode when the infusion pump is connected to the mobile device or in an offline mode when the infusion pump is not connected to the mobile device. For example, this determination may be performed by the operation mode determiner 624 in FIG. 6A.

In accordance with a determination that the infusion pump is in the online mode in the predetermined time period, the processor causes (1260) the infusion pump to deliver a dose of medicament to the user based on the dosing instruction. For example, the dose of medicament may be an optimal insulin dose delivered by the medicament doser 628 in FIG. 6A in an online mode. The optimal insulin dose may be computed based on an insulin dosing target indicated by the dosing instruction.

In accordance with a determination that the infusion pump is in the offline mode in the predetermined time period, the processor performs (1270) the following operations: determining a current pump state corresponding to one of the plurality of predicted pump states, and causes the infusion pump to deliver a dose of medicament to the user based on the offline dosing model and the current pump state. For example, these operations may be performed by the dosing model executor 625 and the medicament doser 628 in FIG. 6A in an offline mode.

Computing devices and other devices discussed herein can include memory. Memory can comprise volatile or non-volatile memory as required by the coupled computing device or processor to not only provide space to execute the instructions or algorithms, but to provide the space to store the instructions themselves. In one embodiment, volatile memory can include random access memory (RAM), dynamic random access memory (DRAM), or static random access memory (SRAM), for example. In one embodiment, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of the disclosure.

In one embodiment, the system or components thereof can comprise or include various modules or engines, each of which is constructed, programmed, configured, or otherwise adapted to autonomously carry out a function or set of functions. The term “engine” as used herein is defined as a real-world device, component, or arrangement of components implemented using hardware, such as by an application specific integrated circuit (ASIC) or field programmable gate array (FPGA), for example, or as a combination of hardware and software, such as by a microprocessor system and a set of program instructions that adapt the engine to implement the particular functionality, which (while being executed) transform the microprocessor system into a special-purpose device.

An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations and should generally not be limited to any particular implementation exemplified herein, unless such limitations are expressly called out. In addition, an engine can itself be composed of more than one sub-engine, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. For example, in an embodiment, each of the processes depicted in FIG. 5 could be implemented within engines as described above. Likewise, in other contemplated embodiments, multiple defined functionalities may be implemented by a single engine that performs those multiple functions, possibly alongside other functions, or distributed differently among a set of engines than specifically illustrated in the examples herein.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that embodiments may comprise fewer features than illustrated in any individual embodiment described above. The embodiments described herein are not meant to be an exhaustive presentation of the ways in which the various features may be combined. Accordingly, the embodiments are not mutually exclusive combinations of features; rather, embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Moreover, reference in the specification to “one embodiment,” “an embodiment,” or “some embodiments” means that a particular feature, structure, or characteristic, described in connection with the embodiment, is included in at least one embodiment of the teaching. The appearances of the phrase “in one embodiment” in various places in the specification are not necessarily all referring to the same embodiment.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein. For purposes of interpreting the claims, it is expressly intended that the provisions of Section 112, sixth paragraph of 35 U.S.C. are not to be invoked unless the specific terms “means for” or “step for” are recited in a claim.

What is claimed is:

1. A system for medicament infusion, the system comprising:
a mobile device associated with a user and configured to:
obtain an online dosing model for dosing determination,
obtain dosing information associated with the user,
generate a dosing instruction for the user based on the online dosing model and the dosing information,
determine a plurality of predicted pump states in a predetermined time period based on the dosing information associated with the user,
generate an offline dosing model for the user in the predetermined time period based on the online dosing model and the plurality of predicted pump states, wherein the offline dosing model has a smaller size than the online dosing model; and
an infusion pump associated with the user and configured to:
obtain the offline dosing model from the mobile device,
store the offline dosing model in a local storage of the infusion pump,
determine, in the predetermined time period, whether the infusion pump is in an online mode when the infusion pump is connected to the mobile device or in an offline mode when the infusion pump is not connected to the mobile device,
in accordance with a determination that the infusion pump is in the online mode in the predetermined time period:
obtain the dosing instruction from the mobile device, and
deliver a dose of medicament to the user based on the dosing instruction,
in accordance with a determination that the infusion pump is in the offline mode in the predetermined time period:
determine a current pump state corresponding to one of the plurality of predicted pump states, and
deliver a dose of medicament to the user based on the offline dosing model and the current pump state.

2. The system of claim 1, wherein the dosing information comprises at least one of:
data from a continuous glucose monitor (CGM) associated with the user;
an estimated glucose influx of the user based on the data from the CGM;
a dosing history of the infusion pump regarding the user; or
information about the current pump state of the infusion pump.

3. The system of claim 1, wherein:
the online dosing model is stored in the mobile device or a cloud server connected to the mobile device;
the online dosing model indicates a dosing target for the user based on the dosing information; and
the dosing instruction includes the dosing target when the infusion pump is in the online mode in the predetermined time period.

4. The system of claim 1, wherein:
the offline dosing model comprises a look-up table indicating the plurality of predicted pump states and a plurality of dosing functions corresponding to the plurality of predicted pump states respectively; and
the infusion pump is configured to determine the dose of medicament in the offline mode based at least in part by:
selecting, from the look-up table, a predicted pump state that is closest to the current pump state among the plurality of predicted pump states,
identifying, from the plurality of dosing functions, a dosing function corresponding to the predicted pump state, and
determining the dose of medicament in the offline mode based on the dosing function.

5. The system of claim 4, wherein:
the online dosing model indicates dosing functions corresponding to a set of pump states of the infusion pump;
the plurality of predicted pump states is a subset of the set of pump states; and
the plurality of predicted pump states are selected from the set of pump states to represent most likely pump states in the predetermined time period.

6. The system of claim 1, wherein the mobile device is further configured to:
update the offline dosing model at periodic time intervals or upon a triggering event, wherein the offline dosing model has a size smaller than a size limit associated with the local storage of the infusion pump; and
transmit a most updated version of the offline dosing model to the infusion pump when the infusion pump is connected to the mobile device.

7. The system of claim 6, wherein the mobile device is configured to update the offline dosing model based at least in part by:
determining an updated time period based on the triggering event or a user input of the user, wherein the updated time period is longer than the predetermined time period;
determining a plurality of updated pump states in the updated time period based on the dosing information associated with the user; and
generating an updated offline dosing model for the user in the updated time period based on the online dosing model and the plurality of updated pump states, wherein the updated offline dosing model has a lower fidelity level than the offline dosing model with respect to the online dosing model.

8. The system of claim 6, wherein the mobile device is configured to update the offline dosing model based at least in part by:
determining that the infusion pump switches from the offline mode to the online mode;
determining a last version of the offline dosing model used by the infusion pump during the offline mode;
comparing the online dosing model with the last version of the offline dosing model based on the dose of medicament delivered during the offline mode to generate a comparison result; and
generating an updated offline dosing model for the user in a next time period based on the online dosing model and the comparison result.

9. The system of claim 1, wherein the infusion pump is further configured to:
determine that infusion pump is in an open loop mode when:
the infusion pump is not connected to the mobile device and the current pump state of the infusion pump is unknown or outside the plurality of predicted pump states in the predetermined time period, or
the infusion pump is still not connected to the mobile device after the predetermined time period; and
in accordance with a determination that the infusion pump is in the open loop mode, deliver a fixed and predetermined dose of medicament to the user based on a predetermined profile of the user.

10. The system of claim 9, wherein when the infusion pump is not connected to the mobile device:
the infusion pump switches from the offline mode to the open loop mode in accordance with a determination that a continuous glucose monitor (CGM) associated with the user is disconnected from the infusion pump; and
the infusion pump switches from the open loop mode to the offline mode in accordance with a determination that the CGM is reconnected to the infusion pump.

11. The system of claim 10, wherein:
at least one of the mobile device or the infusion pump is configured to provide to the user, via a user interface, a first notification that the mobile device is disconnected from the infusion pump, when a connection status between the mobile device and the infusion pump is worse than a threshold;
the infusion pump is configured to provide to the user, via a user interface, a second notification that the infusion pump is in the open loop mode, when the infusion pump enters the open loop mode; and
the first notification and the second notification include at least one of: a message on a screen or a vibration in a particular pattern.

12. The system of claim 1, wherein the infusion pump is further configured to:
obtain one or more characteristics of the user, wherein the one or more characteristics comprise: a target blood glucose, a basal rate, a correction factor, and a carb ratio; and
determine a pump state of the infusion pump at each time step based on the dosing information and the one or more characteristics of the user, wherein the offline dosing model is customized to the user based on the one or more characteristics of the user.

13. The system of claim 12, wherein the infusion pump is configured to determine the pump state based at least in part by:
determining a plurality of activity states, wherein the user is in one of the activity states at any given time and capable of transitioning among the activity states from time to time;
determining state scores each representing a probability that the user is in a respective one of the activity states at the time step; and
determining the pump state of the infusion pump based on: the dosing information, the one or more characteristics of the user, and the state scores.

14. The system of claim 1, wherein:
a connection between the mobile device and the infusion pump is based on at least one of: Internet, Wi-Fi, Bluetooth, or near field communication (NFC).

15. An infusion pump comprising a processor and a non-transitory, computer-readable medium storing instructions which, when executed by the processor, cause the infusion pump to:
transmit dosing information associated with a user to a mobile device of the user;
obtain a dosing instruction from the mobile device, wherein the dosing instruction is generated for the user based on an online dosing model and the dosing information;
obtain an offline dosing model from the mobile device, wherein:
the offline dosing model is generated for the user in a predetermined time period based on the online dosing model and a plurality of predicted pump states,
the plurality of predicted pump states is determined for the predetermined time period based on the dosing information associated with the user, and
the offline dosing model has a smaller size than the online dosing model;
store the offline dosing model in a local storage of the infusion pump;
determine, in the predetermined time period, whether the infusion pump is in an online mode when the infusion pump is connected to the mobile device or in an offline mode when the infusion pump is not connected to the mobile device;
in accordance with a determination that the infusion pump is in the online mode in the predetermined time period, deliver a dose of medicament to the user based on the dosing instruction; and
in accordance with a determination that the infusion pump is in the offline mode in the predetermined time period:
determine a current pump state corresponding to one of the plurality of predicted pump states, and
deliver a dose of medicament to the user based on the offline dosing model and the current pump state.

16. The infusion pump of claim 15, wherein the dosing information comprises at least one of:
data from a continuous glucose monitor (CGM) associated with the user;
an estimated glucose influx of the user based on the data from the CGM;
a dosing history of the infusion pump regarding the user; or
information about the current pump state of the infusion pump.

17. The infusion pump of claim 15, wherein:
the offline dosing model comprises a look-up table indicating the plurality of predicted pump states and a plurality of dosing functions corresponding to the plurality of predicted pump states respectively; and
the infusion pump is configured to determine the dose of medicament in the offline mode based at least in part by:
selecting, from the look-up table, a predicted pump state that is closest to the current pump state among the plurality of predicted pump states,
identifying, from the plurality of dosing functions, a dosing function corresponding to the predicted pump state, and
determining the dose of medicament in the offline mode based on the dosing function.

18. The infusion pump of claim 17, wherein:
the online dosing model indicates dosing functions corresponding to a set of pump states of the infusion pump;
the plurality of predicted pump states is a subset of the set of pump states; and
the plurality of predicted pump states are selected from the set of pump states to represent most likely pump states in the predetermined time period.

19. The infusion pump of claim 15, wherein the instructions, when executed by the processor, further cause the infusion pump to:
determine that infusion pump is in an open loop mode when:
the infusion pump is not connected to the mobile device and the current pump state of the infusion pump is unknown or outside the plurality of predicted pump states in the predetermined time period, or
the infusion pump is still not connected to the mobile device after the predetermined time period; and
in accordance with a determination that the infusion pump is in the open loop mode, deliver a fixed and predetermined dose of medicament to the user based on a predetermined profile of the user.

20. A computer-implemented method for operation of an infusion pump, the method comprising:
transmitting dosing information associated with a user to a mobile device of the user;
obtaining a dosing instruction from the mobile device, wherein the dosing instruction is generated for the user based on an online dosing model and the dosing information;
obtaining an offline dosing model from the mobile device, wherein:
the offline dosing model is generated for the user in a predetermined time period based on the online dosing model and a plurality of predicted pump states,
the plurality of predicted pump states is determined for the predetermined time period based on the dosing information associated with the user, and
the offline dosing model has a smaller size than the online dosing model;
store the offline dosing model in a local storage of the infusion pump;
determining, in the predetermined time period, whether the infusion pump is in an online mode when the infusion pump is connected to the mobile device or in an offline mode when the infusion pump is not connected to the mobile device;
in accordance with a determination that the infusion pump is in the online mode in the predetermined time period, delivering a dose of medicament to the user based on the dosing instruction; and
in accordance with a determination that the infusion pump is in the offline mode in the predetermined time period:
determining a current pump state corresponding to one of the plurality of predicted pump states, and
delivering a dose of medicament to the user based on the offline dosing model and the current pump state.

21. The computer-implemented method of claim 20, further comprising:
determining that infusion pump is in an open loop mode when:
the infusion pump is not connected to the mobile device and the current pump state of the infusion pump is unknown or outside the plurality of predicted pump states in the predetermined time period, or
the infusion pump is still not connected to the mobile device after the predetermined time period; and
in accordance with a determination that the infusion pump is in the open loop mode, delivering a fixed and predetermined dose of medicament to the user based on a predetermined profile of the user.

22. The computer-implemented method of claim 21, further comprising:
switching the infusion pump from the offline mode to the open loop mode in accordance with a determination that a continuous glucose monitor (CGM) associated with the user is disconnected from the infusion pump; and
switching the infusion pump from the open loop mode to the offline mode in accordance with a determination that the CGM is reconnected to the infusion pump.

23. The computer-implemented method of claim 21, further comprising:
obtaining one or more characteristics of the user, wherein the one or more characteristics comprise: a target blood glucose, a basal rate, a correction factor, and a carb ratio; and
determining a pump state of the infusion pump at each time step based on the dosing information and the one or more characteristics of the user, wherein the offline dosing model is customized to the user based on the one or more characteristics of the user.

24. A mobile device comprising a processor and a non-transitory, computer-readable medium storing instructions which, when executed by the processor, cause the mobile device to:
obtain an online dosing model for dosing determination;
obtain dosing information from an infusion pump that is configured for delivering medicament to a user associated with the mobile device, wherein the dosing information indicates, in a predetermined time period, whether the infusion pump is in an online mode when the infusion pump is connected to the mobile device or in an offline mode when the infusion pump is not connected to the mobile device;
generate a dosing instruction for the user based on the online dosing model and the dosing information;
determine a plurality of predicted pump states in the predetermined time period based on the dosing information associated with the user;
generate an offline dosing model for the user in the predetermined time period based on the online dosing model and the plurality of predicted pump states, wherein the offline dosing model has a smaller size than the online dosing model; and
transmit the dosing instruction and the offline dosing model to the infusion pump, wherein the infusion pump is configured to:
store the offline dosing model in a local storage of the infusion pump,
in accordance with a determination that the infusion pump is in the online mode in the predetermined time period, deliver a dose of medicament to the user based on the dosing instruction, and
in accordance with a determination that the infusion pump is in the offline mode in the predetermined time period:
determine a current pump state corresponding to one of the plurality of predicted pump states, and
deliver a dose of medicament to the user based on the offline dosing model and the current pump state.

25. The mobile device of claim 24, wherein the instructions, when executed by the processor, further cause the mobile device to:
update the offline dosing model at periodic time intervals or upon a triggering event, wherein the offline dosing model has a size smaller than a size limit associated with the local storage of the infusion pump; and
transmit a most updated version of the offline dosing model to the infusion pump when the infusion pump is connected to the mobile device.

26. The mobile device of claim 25, wherein the mobile device is configured to update the offline dosing model based at least in part by:
determining an updated time period based on the triggering event or a user input of the user, wherein the updated time period is longer than the predetermined time period;
determining a plurality of updated pump states in the updated time period based on the dosing information associated with the user; and
generating an updated offline dosing model for the user in the updated time period based on the online dosing model and the plurality of updated pump states, wherein the updated offline dosing model has a lower fidelity level than the offline dosing model with respect to the online dosing model.

27. The mobile device of claim 25, wherein the mobile device is configured to update the offline dosing model based at least in part by:
determining that the infusion pump switches from the offline mode to the online mode;
determining a last version of the offline dosing model used by the infusion pump during the offline mode;
comparing the online dosing model with the last version of the offline dosing model based on the dose of medicament delivered during the offline mode to generate a comparison result; and
generating an updated offline dosing model for the user in a next time period based on the online dosing model and the comparison result.

28. A non-transitory, computer-readable medium storing instructions which, when executed by a processor of an electronic device, cause the electronic device to:
transmit dosing information associated with a user to a mobile device of the user;
obtain a dosing instruction from the mobile device, wherein the dosing instruction is generated for the user based on an online dosing model and the dosing information;
obtain an offline dosing model from the mobile device, wherein:
the offline dosing model is generated for the user in a predetermined time period based on the online dosing model and a plurality of predicted pump states,
the plurality of predicted pump states is determined for the predetermined time period based on the dosing information associated with the user, and
the offline dosing model has a smaller size than the online dosing model;
store the offline dosing model in a local storage of the electronic device;
determine, in the predetermined time period, whether the electronic device is in an online mode when the electronic device is connected to the mobile device or in an offline mode when the electronic device is not connected to the mobile device;
in accordance with a determination that the electronic device is in the online mode in the predetermined time period, deliver a dose of medicament to the user based on the dosing instruction; and
in accordance with a determination that the electronic device is in the offline mode in the predetermined time period:
determine a current pump state corresponding to one of the plurality of predicted pump states, and
deliver a dose of medicament to the user based on the offline dosing model and the current pump state.
